(19) United States
(12) Patent Application Publication
(10) Pub. No.: US 2023/0070842 A1
(43) Pub. Date: Mar. 9, 2023

(54) SYSTEMS AND METHODS OF SEMANTIC TAGGING

(71) Applicant: Johnson Controls Tyco IP Holdings LLP, Milwaukee, WI (US)

(72) Inventor: Wang Wei, Beijing City (CN)

(73) Assignee: Johnson Controls Tyco IP Holdings LLP, Milwaukee, WI (US)

(21) Appl. No.: 17/510,062

(22) Filed: Oct. 25, 2021

(30) Foreign Application Priority Data
Sep. 6, 2021 (CN) PCT/CN2021/116652

(51) Int. Cl. G16H 40/20; G16H 40/40; G06F 16/908; G06F 40/30; G16H 10/60

(52) U.S. Cl. CPC G16H 40/20; G16H 40/40; G06F 16/908; G06F 40/30; G16H 10/60

(57) ABSTRACT

A method of retrieving data using metadata tags including identifying, by a processing circuit from a data structure, a digital representation of a device deployed within a space, tagging, by the processing circuit, a data point associated with the digital representation of the device with a semantic description having a tag schema, receiving, by the processing circuit, a query including a partial string referencing the tag schema, identifying, by the processing circuit, the semantic description from a plurality of semantic descriptions based on the partial string of the query and the tag schema, retrieving, by the processing circuit, one or more tagged data points by querying the data structure using the semantic description, and automatically performing an operation using the one or more tagged data points.

### CROSS-REFERENCE TO RELATED PATENT APPLICATIONS

This application claims the benefit of and priority to International Patent Application No. PCT/CN2021/116652, filed Sep. 6, 2021, the entire disclosure of which is incorporated by reference herein.

### BACKGROUND

The present disclosure relates generally to building systems that manage a building. The present disclosure relates more particularly to tagging control data within a building automation system (BAS).

### SUMMARY

One embodiment of the present disclosure is a method of embedding trend data in a user interface comprising tagging a data point stored in a data structure with a semantic description, receiving a query from a user including at least a partial string referencing the semantic description, retrieving, from the data structure according to the semantic description of the query, a tagged data point, generating a user interface element to display real time trend data associated with the retrieved tagged data point, and automatically embedding the user interface element in a user interface.

In some embodiments, the real time trend data includes at least one of an alarm status or a sensor measurement value. In some embodiments, the data structure includes an electronic medical record (EMR) and wherein the real time trend data includes at least one of a patient health status or a biological measurement associated with a patient. In some embodiments, the data point is stored using a resource description framework (RDF) format. In some embodiments, retrieving the tagged data point, generating the user interface element, and automatically embedding the user interface element are performed automatically in response to receiving the query. In some embodiments, the method further comprises automatically formatting at least one of a units or a display scale of the user interface element based on the real time trend data. In some embodiments, the data structure includes a digital twin representing at least one of a space, a person, a piece of equipment, or an event. In some embodiments, the data structure is a graph data structure.

Another embodiment of the present disclosure is a controller for managing building equipment comprising a processing circuit including a processor and memory, the memory storing instructions thereon that, when executed by the processor, causes the processing circuit to tag a data point stored in a data structure with a semantic description, receive a query from a user including at least a partial string referencing the semantic description, retrieve, from the data structure according to the semantic description of the query, a tagged data point, generate a user interface element to display real time trend data associated with the retrieved tagged data point, and automatically embed the user interface element in a user interface.

In some embodiments, the real time trend data includes at least one of an alarm status or a sensor measurement value. In some embodiments, the data point is stored using a resource description framework (RDF) format. In some embodiments, retrieving the tagged data point, generating the user interface element, and automatically embedding the user interface element are performed automatically in response to receiving the query. In some embodiments, the instructions further cause the processing circuit to automatically format at least one of a units or a display scale of the user interface element based on the real time trend data. In some embodiments, the data structure includes a digital twin representing at least one of a space, a person, a piece of equipment, or an event. In some embodiments, the data structure is a graph data structure.

Another embodiment of the present disclosure is one or more non-transitory computer-readable storage media having instructions stored thereon that, when executed by one or more processors, cause the one or more processors to tag a data point stored in a data structure with a semantic description, receive a query from a user including at least a partial string referencing the semantic description, retrieve, from the data structure according to the semantic description of the query, a tagged data point, generate a user interface element to display real time trend data associated with the retrieved tagged data point, and automatically embed the user interface element in a user interface.

In some embodiments, the real time trend data includes at least one of an alarm status or a sensor measurement value. In some embodiments, the data structure includes an electronic medical record (EMR) and wherein the real time trend data includes at least one of a patient health status or a biological measurement associated with a patient. In some embodiments, the data point is stored using a resource description framework (RDF) format. In some embodiments, retrieving the tagged data point, generating the user interface element, and automatically embedding the user interface element are performed automatically in response to receiving the query.

Another embodiment of the present disclosure is a method of retrieving data using metadata tags including identifying, by a processing circuit from a data structure, a digital representation of a device deployed within a space, tagging, by the processing circuit, a data point associated with the digital representation of the device with a semantic description having a tag schema, receiving, by the processing circuit, a query including a partial string referencing the tag schema, identifying, by the processing circuit, the semantic description from a plurality of semantic descriptions based on the partial string of the query and the tag schema, retrieving, by the processing circuit, one or more tagged data points by querying the data structure using the semantic description, and automatically performing an operation using the one or more tagged data points.

In some embodiments, automatically performing the operation includes generating, by the processing circuit, a user interface element to display real time trend data associated with the retrieved one or more tagged data points, and automatically embedding, by the processing circuit, the user interface element in a user interface. In some embodiments, the real time trend data includes at least one of an alarm status or a sensor measurement value. In some embodiments, the data structure includes an electronic medical record (EMR) and wherein the real time trend data includes at least one of a patient health status or a biological measurement associated with a patient. In some embodiments, retrieving the one or more tagged data points, generating the user interface element, and automatically embedding the user interface element are performed automatically in response to identifying the semantic description. In some embodiments, the method further comprises automatically formatting, by the processing circuit, at least one of a units or a display scale of the user interface element based on the real time trend data. In some embodiments, the data point is stored using a resource description framework (RDF) format. In some embodiments, the data structure includes a digital twin representing at least one of a space, a person, a piece of equipment, or an event. In some embodiments, automatically performing the operation includes at least one of: (i) determining a fault associated with the space based on the one or more tagged data points, (ii) generating a predictive control model for the space based on the one or more tagged data points, (iii) generating a control message to control the device based on the one or more tagged data points, (iv) controlling an energy usage associated with the space based on the one or more tagged data points, (v) training a machine learning model associated with the space using the one or more tagged data points, (vi) updating a model associated with the space based on user feedback corresponding to the one or more tagged data points, or (vii) updating an architectural model for the space based on the one or more tagged data points.

### BRIEF DESCRIPTION OF THE DRAWINGS

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1 is a drawing of a building equipped with a HVAC system, according to an exemplary embodiment.

FIG. 2 is a block diagram of a building automation system (BAS) that may be used to monitor and/or control the building of FIG. 1, according to an exemplary embodiment.

FIGS. 3A-3B illustrate a data structure including tagged entities, according to various exemplary embodiments.

FIG. 4 is a graphical user interface (GUI) to facilitate semantic tagging, according to an exemplary embodiment.

FIGS. 5A-5B illustrate a GUI for tagging entities and/or data points, according to various exemplary embodiments.

FIG. 6 is a GUI for executing queries based on semantic tags, according to an exemplary embodiment.

FIG. 7 is a GUI element associated with a semantic tag, according to an exemplary embodiment.

FIG. 8 is a flowchart illustrating a method of tagging a data point, according to an exemplary embodiment.

FIG. 9 is a flowchart illustrating a method of serving an analytics element associated with a tag, according to an exemplary embodiment.

FIG. 10 is graph data structure for digitally representing entities, according to an exemplary embodiment.

### DETAILED DESCRIPTION

### Overview

Referring generally to the FIGURES, systems and methods of semantic tagging are disclosed. Specifically, systems and methods of the present disclosure may facilitate modifying data structure elements to include semantic tags and generating graphical user interface (GUI) elements associated with the semantic tags. In various embodiments, data structures such as those representing building data or healthcare data include metadata such as semantic tags. For example, a graph data structure representing a digital twin of a building may include metadata that conforms to the Brick Schema from the Brick Consortium, Inc. However, often there are many choices to choose from when applying metadata to a data structure. For example, in a building automation context, an operator may have to choose from 1,500 equipment types when adding metadata to a digital representation of a piece of HVAC equipment. As another example, in a healthcare context, a healthcare provider may have to choose from hundreds of diagnoses when adding metadata to a digital representation of an individual. It may be prohibitively difficult and/or time consuming to manually identify a tag for an entity such as a space, a piece of equipment, a person, or an event. For example, in many scenarios an operator may skip adding semantic tags for a new piece of equipment when creating a digital representation of the new piece of equipment because the appropriate semantic tags are too difficult to identify (e.g., from a scrolling list of tags, etc.). In various embodiments, a BAS functionality may be limited as a result of missing metadata such as tags. Therefore, there is a need for systems and methods to facilitate semantic tagging of entities within a data structure such as a BAS or an electronic medical record (EMR).

Systems and methods of the present disclosure may solve these shortcomings by facilitating semantic tagging of entities. For example, systems and methods of the present disclosure may generate metadata suggestions such as suggested tags for an entity based on a name of the entity. As another example, systems and methods of the present disclosure may facilitate generating tag suggestions based on keywords. For example, a user may enter “RATS” and systems and methods of the present disclosure may suggest “Return Air Temperature Sensor” as a tag based on the user's input. In various embodiments, systems and methods of the present disclosure facilitate auto-tagging of entities. For example, systems and methods of the present disclosure may automatically tag (e.g., with little to no user input, etc.) a digital representation of an HVAC component based on context information associated with the digital representation (e.g., which entities the HVAC component is related to, the type of data associated with the HVAC component, etc.).

In various embodiments, systems and methods of the present disclosure facilitate querying and/or generating GUI elements based on tagged entities. For example, a user may generate a query to quickly identify the entities in a data structure having the tag “Return Air Temperature Sensor.” As another example, the user may generate a GUI element such as a dial to illustrate one or more parameters associated with a tagged entity such as a sensor value associated with a tagged air temperature sensor.

Additionally or alternatively, systems and methods of the present disclosure may facilitate performing operations using retrieve tagged data points. For example, a user may generate a query to quickly identify a number of sensors associated with a space and automatically feed the identified sensors into a fault prediction system to determine the presence/absence of a fault associated with the space. As another example, a user may retrieve data points having a tag indicating that the data points are associated with a specific firmware version and may automatically update an AB testing model using the retrieved data points. In some embodiments, the retrieved data points are automatically fed into a machine learning model to train the machine learning model, thereby reducing an amount of manual effort required to identify and segment training data.

### Building Management System and HVAC System

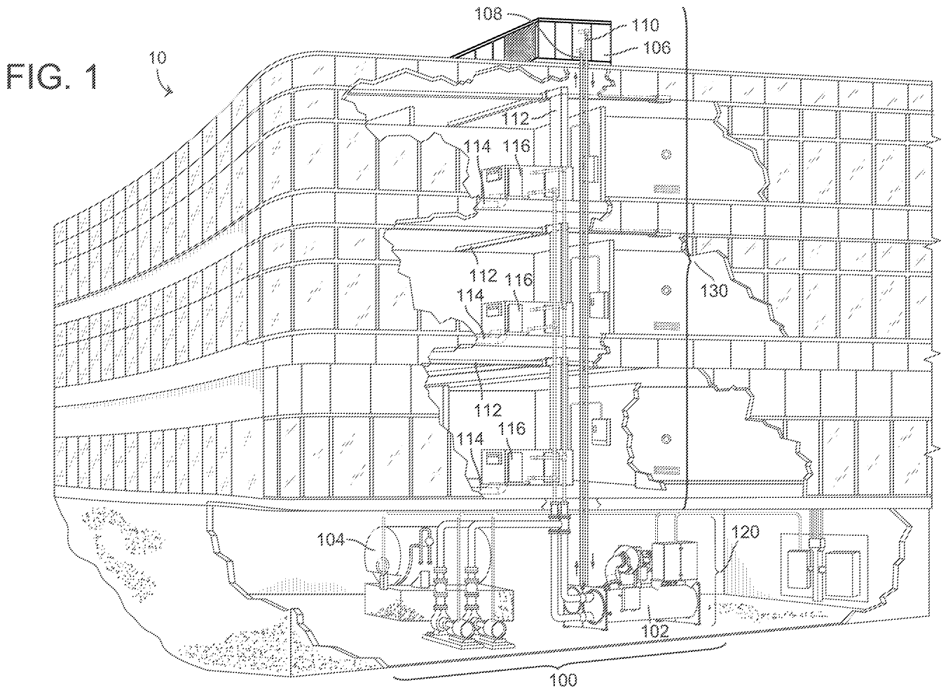

Referring now to FIG. 1, an exemplary building management system (BMS) and HVAC system in which the systems and methods of the present invention can be implemented are shown, according to an exemplary embodiment. Referring particularly to FIG. 1, a perspective view of a building 10 is shown. Building 10 is served by a BMS. A BMS is, in general, a system of devices configured to control, monitor, and manage equipment in or around a building or building area. A BMS can include, for example, a HVAC system, a security system, a lighting system, a fire alerting system, and/or any other system that is capable of managing building functions or devices, or any combination thereof.

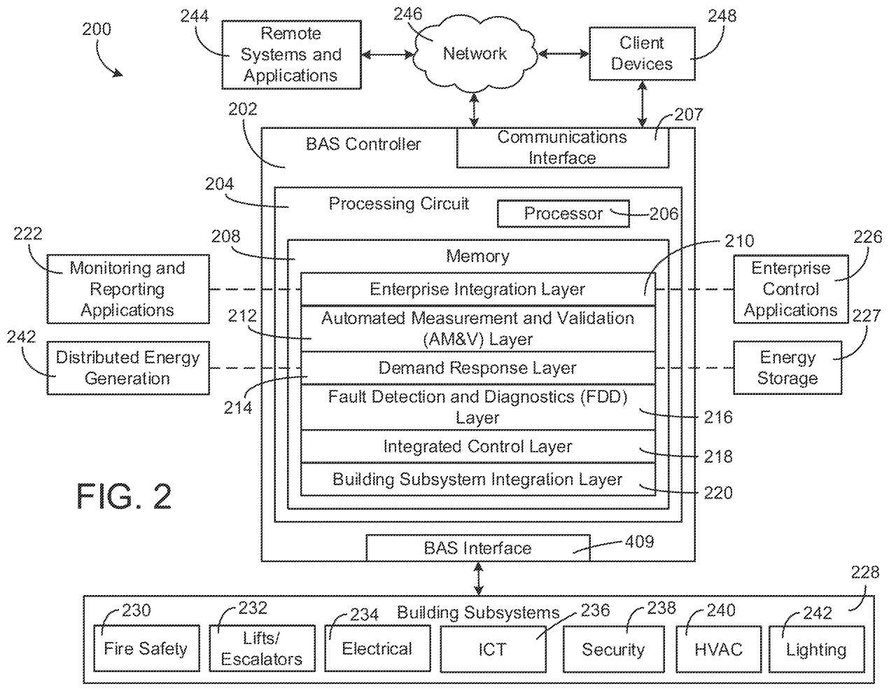

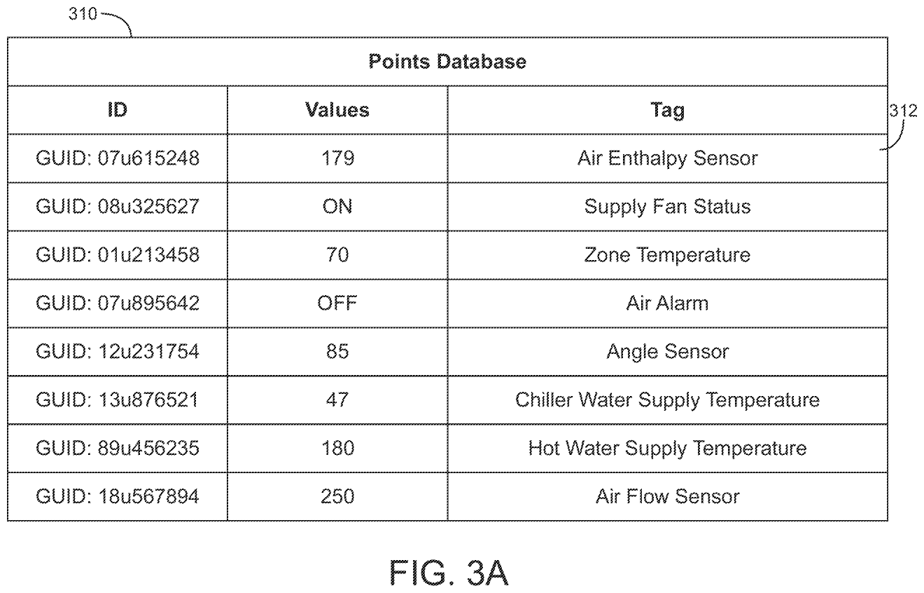

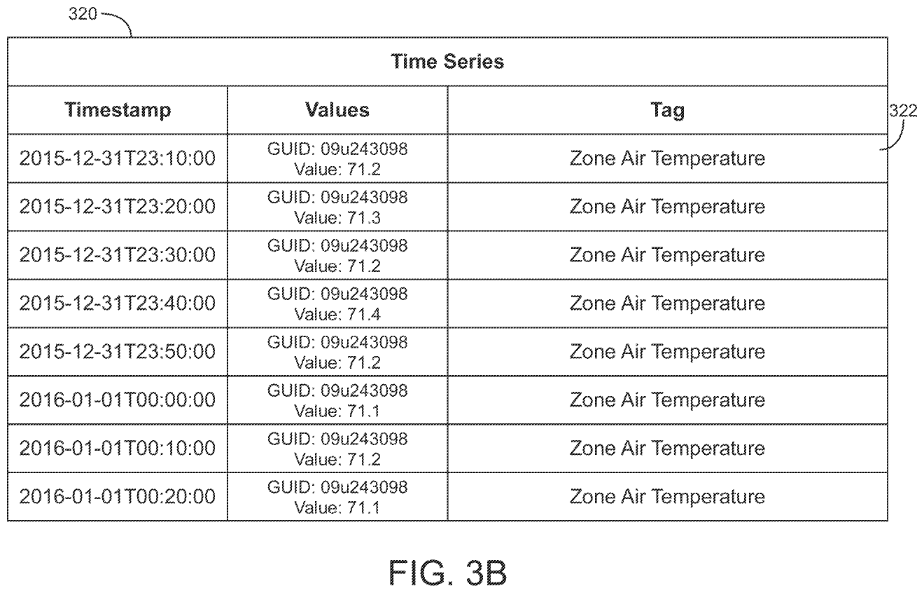

The BMS that serves building 10 includes an HVAC system 100. HVAC system 100 can include a plurality of HVAC devices (e.g., heaters, chillers, air handling units, pumps, fans, thermal energy storage, etc.) configured to provide heating, cooling, ventilation, or other services for building 10. For example, HVAC system 100 is shown to include a waterside system 120 and an airside system 130. Waterside system 120 can provide a heated or chilled fluid to an air handling unit of airside system 130. Airside system 130 can use the heated or chilled fluid to heat or cool an airflow provided to building 10. An exemplary waterside system and airside system which can be used in HVAC system 100 are described in greater detail with reference to FIGS. 2-3.

HVAC system 100 is shown to include a chiller 102, a boiler 104, and a rooftop air handling unit (AHU) 106. Waterside system 120 can use boiler 104 and chiller 102 to heat or cool a working fluid (e.g., water, glycol, etc.) and can circulate the working fluid to AHU 106. In various embodiments, the HVAC devices of waterside system 120 can be located in or around building 10 (as shown in FIG. 1) or at an offsite location such as a central plant (e.g., a chiller plant, a steam plant, a heat plant, etc.). The working fluid can be heated in boiler 104 or cooled in chiller 102, depending on whether heating or cooling is required in building 10. Boiler 104 can add heat to the circulated fluid, for example, by burning a combustible material (e.g., natural gas) or using an electric heating element. Chiller 102 can place the circulated fluid in a heat exchange relationship with another fluid (e.g., a refrigerant) in a heat exchanger (e.g., an evaporator) to absorb heat from the circulated fluid. The working fluid from chiller 102 and/or boiler 104 can be transported to AHU 106 via piping 108.

AHU 106 can place the working fluid in a heat exchange relationship with an airflow passing through AHU 106 (e.g., via one or more stages of cooling coils and/or heating coils). The airflow can be, for example, outside air, return air from within building 10, or a combination of both. AHU 106 can transfer heat between the airflow and the working fluid to provide heating or cooling for the airflow. For example, AHU 106 can include one or more fans or blowers configured to pass the airflow over or through a heat exchanger containing the working fluid. The working fluid can then return to chiller 102 or boiler 104 via piping 110.

Airside system 130 can deliver the airflow supplied by AHU 106 (i.e., the supply airflow) to building 10 via air supply ducts 112 and can provide return air from building 10 to AHU 106 via air return ducts 114. In some embodiments, airside system 130 includes multiple variable air volume (VAV) units 116. For example, airside system 130 is shown to include a separate VAV unit 116 on each floor or zone of building 10. VAV units 116 can include dampers or other flow control elements that can be operated to control an amount of the supply airflow provided to individual zones of building 10. In other embodiments, airside system 130 delivers the supply airflow into one or more zones of building 10 (e.g., via supply ducts 112) without using intermediate VAV units 116 or other flow control elements. AHU 106 can include various sensors (e.g., temperature sensors, pressure sensors, etc.) configured to measure attributes of the supply airflow. AHU 106 can receive input from sensors located within AHU 106 and/or within the building zone and can adjust the flow rate, temperature, or other attributes of the supply airflow through AHU 106 to achieve setpoint conditions for the building zone.

Referring now to FIG. 2, a block diagram of a building automation system (BAS) 200 is shown, according to an exemplary embodiment. BAS 200 can be implemented in building 10 to automatically monitor and control various building functions. BAS 200 is shown to include BAS controller 202 and a plurality of building subsystems 228. Building subsystems 228 are shown to include a building electrical subsystem 234, an information communication technology (ICT) subsystem 236, a security subsystem 238, a HVAC subsystem 240, a lighting subsystem 242, a lift/escalators subsystem 232, and a fire safety subsystem 230. In various embodiments, building subsystems 228 can include fewer, additional, or alternative subsystems. For example, building subsystems 228 can also or alternatively include a refrigeration subsystem, an advertising or signage subsystem, a cooking subsystem, a vending subsystem, a printer or copy service subsystem, or any other type of building subsystem that uses controllable equipment and/or sensors to monitor or control building 10. In some embodiments, building subsystems 228 include a waterside system and/or an airside system. A waterside system and an airside system are described with further reference to U.S. patent application Ser. No. 15/631,830 filed Jun. 23, 2017, the entirety of which is incorporated by reference herein.

Each of building subsystems 228 can include any number of devices, controllers, and connections for completing its individual functions and control activities. HVAC subsystem 240 can include many of the same components as HVAC system 100, as described with reference to FIG. 1. For example, HVAC subsystem 240 can include a chiller, a boiler, any number of air handling units, economizers, field controllers, supervisory controllers, actuators, temperature sensors, and other devices for controlling the temperature, humidity, airflow, or other variable conditions within building 10. Lighting subsystem 242 can include any number of light fixtures, ballasts, lighting sensors, dimmers, or other devices configured to controllably adjust the amount of light provided to a building space. Security subsystem 238 can include occupancy sensors, video surveillance cameras, digital video recorders, video processing servers, intrusion detection devices, access control devices and servers, or other security-related devices.

Still referring to FIG. 2, BAS controller 266 is shown to include a communications interface 207 and a BAS interface 209. Interface 207 can facilitate communications between BAS controller 202 and external applications (e.g., monitoring and reporting applications 222, enterprise control applications 226, remote systems and applications 244, applications residing on client devices 248, etc.) for allowing user control, monitoring, and adjustment to BAS controller 266 and/or subsystems 228. Interface 207 can also facilitate communications between BAS controller 202 and client devices 248. BAS interface 209 can facilitate communications between BAS controller 202 and building subsystems 228 (e.g., HVAC, lighting security, lifts, power distribution, business, etc.).

Interfaces 207, 209 can be or include wired or wireless communications interfaces (e.g., jacks, antennas, transmitters, receivers, transceivers, wire terminals, etc.) for conducting data communications with building subsystems 228 or other external systems or devices. In various embodiments, communications via interfaces 207, 209 can be direct (e.g., local wired or wireless communications) or via a communications network 246 (e.g., a WAN, the Internet, a cellular network, etc.). For example, interfaces 207, 209 can include an Ethernet card and port for sending and receiving data via an Ethernet-based communications link or network. In another example, interfaces 207, 209 can include a Wi-Fi transceiver for communicating via a wireless communications network. In another example, one or both of interfaces 207, 209 can include cellular or mobile phone communications transceivers. In one embodiment, communications interface 207 is a power line communications interface and BAS interface 209 is an Ethernet interface. In other embodiments, both communications interface 207 and BAS interface 209 are Ethernet interfaces or are the same Ethernet interface.

Still referring to FIG. 2, BAS controller 202 is shown to include a processing circuit 204 including a processor 206 and memory 208. Processing circuit 204 can be communicably connected to BAS interface 209 and/or communications interface 207 such that processing circuit 204 and the various components thereof can send and receive data via interfaces 207, 209. Processor 206 can be implemented as a general purpose processor, an application specific integrated circuit (ASIC), one or more field programmable gate arrays (FPGAs), a group of processing components, or other suitable electronic processing components.

Memory 208 (e.g., memory, memory unit, storage device, etc.) can include one or more devices (e.g., RAM, ROM, Flash memory, hard disk storage, etc.) for storing data and/or computer code for completing or facilitating the various processes, layers and modules described in the present application. Memory 208 can be or include volatile memory or non-volatile memory. Memory 208 can include database components, object code components, script components, or any other type of information structure for supporting the various activities and information structures described in the present application. According to an exemplary embodiment, memory 208 is communicably connected to processor 206 via processing circuit 204 and includes computer code for executing (e.g., by processing circuit 204 and/or processor 206) one or more processes described herein.

In some embodiments, BAS controller 202 is implemented within a single computer (e.g., one server, one housing, etc.). In various other embodiments BAS controller 202 can be distributed across multiple servers or computers (e.g., that can exist in distributed locations). Further, while FIG. 2 shows applications 222 and 226 as existing outside of BAS controller 202, in some embodiments, applications 222 and 226 can be hosted within BAS controller 202 (e.g., within memory 208).

Still referring to FIG. 2, memory 208 is shown to include an enterprise integration layer 210, an automated measurement and validation (AM&V) layer 212, a demand response (DR) layer 214, a fault detection and diagnostics (FDD) layer 216, an integrated control layer 218, and a building subsystem integration later 220. Layers 210-220 is configured to receive inputs from building subsystems 228 and other data sources, determine optimal control actions for building subsystems 228 based on the inputs, generate control signals based on the optimal control actions, and provide the generated control signals to building subsystems 228 in some embodiments. The following paragraphs describe some of the general functions performed by each of layers 210-220 in BAS 200.

Enterprise integration layer 210 can be configured to serve clients or local applications with information and services to support a variety of enterprise-level applications. For example, enterprise control applications 226 can be configured to provide subsystem-spanning control to a graphical user interface (GUI) or to any number of enterprise-level business applications (e.g., accounting systems, user identification systems, etc.). Enterprise control applications 226 can also or alternatively be configured to provide configuration GUIs for configuring BAS controller 202. In yet other embodiments, enterprise control applications 226 can work with layers 210-220 to optimize building performance (e.g., efficiency, energy use, comfort, or safety) based on inputs received at interface 207 and/or BAS interface 209.

Building subsystem integration layer 220 can be configured to manage communications between BAS controller 202 and building subsystems 228. For example, building subsystem integration layer 220 can receive sensor data and input signals from building subsystems 228 and provide output data and control signals to building subsystems 228. Building subsystem integration layer 220 can also be configured to manage communications between building subsystems 228. Building subsystem integration layer 220 translate communications (e.g., sensor data, input signals, output signals, etc.) across a plurality of multi-vendor/multi-protocol systems.

Demand response layer 214 can be configured to optimize resource usage (e.g., electricity use, natural gas use, water use, etc.) and/or the monetary cost of such resource usage in response to satisfy the demand of building 10. The optimization can be based on time-of-use prices, curtailment signals, energy availability, or other data received from utility providers, distributed energy generation systems 224, from energy storage 227, or from other sources. Demand response layer 214 can receive inputs from other layers of BAS controller 202 (e.g., building subsystem integration layer 220, integrated control layer 218, etc.). The inputs received from other layers can include environmental or sensor inputs such as temperature, carbon dioxide levels, relative humidity levels, air quality sensor outputs, occupancy sensor outputs, room schedules, and the like. The inputs can also include inputs such as electrical use (e.g., expressed in kWh), thermal load measurements, pricing information, projected pricing, smoothed pricing, curtailment signals from utilities, and the like.

According to an exemplary embodiment, demand response layer 214 includes control logic for responding to the data and signals it receives. These responses can include communicating with the control algorithms in integrated control layer 218, changing control strategies, changing setpoints, or activating/deactivating building equipment or subsystems in a controlled manner. Demand response layer 214 can also include control logic configured to determine when to utilize stored energy. For example, demand response layer 214 can determine to begin using energy from energy storage 227 just prior to the beginning of a peak use hour.

In some embodiments, demand response layer 214 includes a control module configured to actively initiate control actions (e.g., automatically changing setpoints) which minimize energy costs based on one or more inputs representative of or based on demand (e.g., price, a curtailment signal, a demand level, etc.). In some embodiments, demand response layer 214 uses equipment models to determine an optimal set of control actions. The equipment models can include, for example, thermodynamic models describing the inputs, outputs, and/or functions performed by various sets of building equipment. Equipment models can represent collections of building equipment (e.g., subplants, chiller arrays, etc.) or individual devices (e.g., individual chillers, heaters, pumps, etc.).

Demand response layer 214 can further include or draw upon one or more demand response policy definitions (e.g., databases, XML, files, etc.). The policy definitions can be edited or adjusted by a user (e.g., via a graphical user interface) so that the control actions initiated in response to demand inputs can be tailored for the user's application, desired comfort level, particular building equipment, or based on other concerns. For example, the demand response policy definitions can specify which equipment can be turned on or off in response to particular demand inputs, how long a system or piece of equipment should be turned off, what setpoints can be changed, what the allowable set point adjustment range is, how long to hold a high demand setpoint before returning to a normally scheduled setpoint, how close to approach capacity limits, which equipment modes to utilize, the energy transfer rates (e.g., the maximum rate, an alarm rate, other rate boundary information, etc.) into and out of energy storage devices (e.g., thermal storage tanks, battery banks, etc.), and when to dispatch on-site generation of energy (e.g., via fuel cells, a motor generator set, etc.).

Integrated control layer 218 can be configured to use the data input or output of building subsystem integration layer 220 and/or demand response later 214 to make control decisions. Due to the subsystem integration provided by building subsystem integration layer 220, integrated control layer 218 can integrate control activities of the subsystems 228 such that the subsystems 228 behave as a single integrated supersystem. In an exemplary embodiment, integrated control layer 218 includes control logic that uses inputs and outputs from a plurality of building subsystems to provide greater comfort and energy savings relative to the comfort and energy savings that separate subsystems could provide alone. For example, integrated control layer 218 can be configured to use an input from a first subsystem to make an energy-saving control decision for a second subsystem. Results of these decisions can be communicated back to building subsystem integration layer 220.

Integrated control layer 218 is shown to be logically below demand response layer 214. Integrated control layer 218 can be configured to enhance the effectiveness of demand response layer 214 by enabling building subsystems 228 and their respective control loops to be controlled in coordination with demand response layer 214. This configuration can reduce disruptive demand response behavior relative to conventional systems. For example, integrated control layer 218 can be configured to assure that a demand response-driven upward adjustment to the setpoint for chilled water temperature (or another component that directly or indirectly affects temperature) does not result in an increase in fan energy (or other energy used to cool a space) that would result in greater total building energy use than was saved at the chiller.

Integrated control layer 218 can be configured to provide feedback to demand response layer 214 so that demand response layer 214 checks that constraints (e.g., temperature, lighting levels, etc.) are properly maintained even while demanded load shedding is in progress. The constraints can also include setpoint or sensed boundaries relating to safety, equipment operating limits and performance, comfort, fire codes, electrical codes, energy codes, and the like. Integrated control layer 218 is also logically below fault detection and diagnostics layer 216 and automated measurement and validation layer 212. Integrated control layer 218 can be configured to provide calculated inputs (e.g., aggregations) to these higher levels based on outputs from more than one building subsystem.

Automated measurement and validation (AM&V) layer 212 can be configured to verify that control strategies commanded by integrated control layer 218 or demand response layer 214 are working properly (e.g., using data aggregated by AM&V layer 212, integrated control layer 218, building subsystem integration layer 220, FDD layer 216, or otherwise). The calculations made by AM&V layer 212 can be based on building system energy models and/or equipment models for individual BAS devices or subsystems. For example, AM&V layer 212 can compare a model-predicted output with an actual output from building subsystems 228 to determine an accuracy of the model.

Fault detection and diagnostics (FDD) layer 216 can be configured to provide on-going fault detection for building subsystems 228, building subsystem devices (i.e., building equipment), and control algorithms used by demand response layer 214 and integrated control layer 218. FDD layer 216 can receive data inputs from integrated control layer 218, directly from one or more building subsystems or devices, or from another data source. FDD layer 216 can automatically diagnose and respond to detected faults. The responses to detected or diagnosed faults can include providing an alarm message to a user, a maintenance scheduling system, or a control algorithm configured to attempt to repair the fault or to work-around the fault.

FDD layer 216 can be configured to output a specific identification of the faulty component or cause of the fault (e.g., loose damper linkage) using detailed subsystem inputs available at building subsystem integration layer 220. In other exemplary embodiments, FDD layer 216 is configured to provide “fault” events to integrated control layer 218 which executes control strategies and policies in response to the received fault events. According to an exemplary embodiment, FDD layer 216 (or a policy executed by an integrated control engine or business rules engine) can shut-down systems or direct control activities around faulty devices or systems to reduce energy waste, extend equipment life, or assure proper control response.

FDD layer 216 can be configured to store or access a variety of different system data stores (or data points for live data). FDD layer 216 can use some content of the data stores to identify faults at the equipment level (e.g., specific chiller, specific AHU, specific terminal unit, etc.) and other content to identify faults at component or subsystem levels. For example, building subsystems 228 can generate temporal (i.e., time-series) data indicating the performance of BAS 200 and the various components thereof. The data generated by building subsystems 228 can include measured or calculated values that exhibit statistical characteristics and provide information about how the corresponding system or process (e.g., a temperature control process, a flow control process, etc.) is performing in terms of error from its setpoint. These processes can be examined by FDD layer 216 to expose when the system begins to degrade in performance and alarm a user to repair the fault before it becomes more severe.

### Semantic Tagging

Referring now to FIG. 3A, data structure 310 including tagged data points is shown, according to an exemplary embodiment. In various embodiments, data structure 310 is a table. In some embodiments, data structure 310 may represent tagged entities. In some embodiments, data structure 310 may be represented as a graph data structure. For example, data structure 310 may include a graph data structure having nodes corresponding to each entity and connections between nodes representing relationships between entities. In various embodiments, data structure 310 includes one or more entities such as spaces, equipment, devices, people, events, and/or the like. In various embodiments, entities may include identifiers such as alphanumeric strings.

Each data point and/or entity may have associated metadata such as tags 312. Tags 312 may include semantic descriptions of data points and/or entities such as an equipment type, a disease diagnosis, a data type, an associated space, and/or the like. In various embodiments, tags 312 are associated with connections between nodes in a graph data structure. For example, tags 312 may represent relationships between entities such as a tag indicating that a particular VAV unit serves a particular room in a building. In various embodiments, tags 312 may be manually assigned by a user. For example, when a new piece of building equipment is added to a building a user may manually assign tags to a digital representation of the building equipment within a BAS. In various embodiments, manually assigning tags and/or assigning tags without assistance is prohibitively difficult. For example, a building may have hundreds of types of sensors and identifying the correct sensor type from a list to manually add to a digital representation of a sensor may be time consuming. In some embodiments, acronyms and/or quick references may be used to facilitate efficient lookup of tags 312. For example, a tag “2701FCU101 1-13N7E OFFICE DA-T” may be found using “27” as a site name, “01” as a building number, “FCU101” as a device or a system name, “1-13N7E” as a device location, “OFFICE” as a space type, and/or “DA-T” as a discharge air temperature. In some embodiments, tags 312 are identified based on METASYS, BACnet, or BIM acronyms.

As shown, data structure 310 includes an ID column, a values column, and a semantic data tag column. Data structure 310 may include rows. Each row may represent a data point and may include values (e.g., strings) within each column. The data points may be associated with values generated and/or measured by sensors associated with an entity. In some embodiments, data points are associated with a device identifier and value. The device identifier may indicate a device associated with the data point. For example, the device identifier may include “GUID: 07u615248.” The value may indicate the value that a sensor measured and/or generated. In some embodiments, although not shown, data points may be associated with a timestamp indicating a time that the sensor measured and/or generated the value or a time that a database received the data point. In some embodiments, data structure 310 includes untagged data (not shown). Systems and methods of the present disclosure may facilitate tagging untagged data points and/or entities.

Referring now to FIG. 3B, data structure 320 including tagged timeseries data is shown, according to an exemplary embodiment. In various embodiments, data structure 320 is a table. In some embodiments, data structure 320 may be represented as a matrix. For example, data structure 320 may include a matrix having timeseries data associated with entities of a digital twin. The timeseries data may be associated with sensor measurements such as an air temperature sensor. In various embodiments, the timeseries data may have associated metadata such as tags 322. Tags 322 may include semantic descriptions of the timeseries data such as a data type, an associated space, a state description (e.g., whether the data corresponds to a safe/unsafe state of a space, etc.), and/or the like. In some embodiments, timeseries data is tagged according to an entity that produced the timeseries data. For example, timeseries data generated by a temperature sensor that monitors a zone air temperature may be tagged as zone air temperature data.

Data structure 320 may include timeseries data points. Each data point may include any of a timestamp, a value, and a semantic tag identifying different aspects (e.g., points or characteristics of a building) that the data point is associated with. The timestamps may be associated with a time that the data point was generated or a time that a database received the data point. Tags 322 may include semantic data tags associated with the data points that describe what aspect or characteristic of a building that the values of the data point are associated with. Tags 322 may be associated with the data points by a building management system that provided the data points, by an administrator input, and/or by a system as described herein.

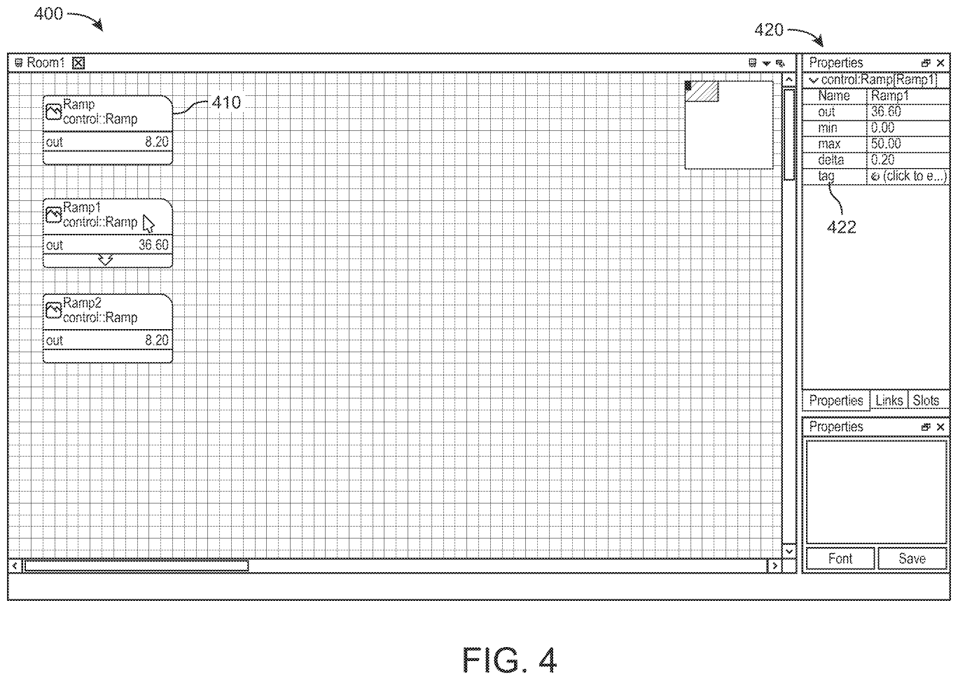

Referring now to FIG. 4, GUI 400 for tagging entities is shown, according to an exemplary embodiment. GUI 400 may be generated by BAS controller 202 and displayed on a device such as client devices 248. In various embodiments, GUI 400 facilitates identifying tags associated with entities and/or tagging entities. For example, GUI 400 may facilitate surfacing tags for a digital representation of an entity such as a piece of building equipment based on acronyms and/or quick references associated with the piece of building equipment and/or the tag. In various embodiments, GUI 400 includes element 410 representing an entity such as an air temperature sensor. In various embodiments, the entity may be associated with data such as sensor data. In various embodiments, additional data such as metadata is associated with the entity via properties 420. For example, properties 420 may include a name of the entity, a current output of the entity (e.g., a sensor output, etc.), a minimum output of the entity (e.g., over some time period, etc.), a maximum output of the entity, a statistical measure such as a variance or a delta, and/or tag 422. In various embodiments, users may assign tag 422 to an entity represented by element 410 using GUI 400. For example, a user may select element 410 and click tag 422 to assign a tag to an entity represented by element 410. In various embodiments, systems and methods of the present disclosure facilitate a user assigning tag 422 to an entity as described below.

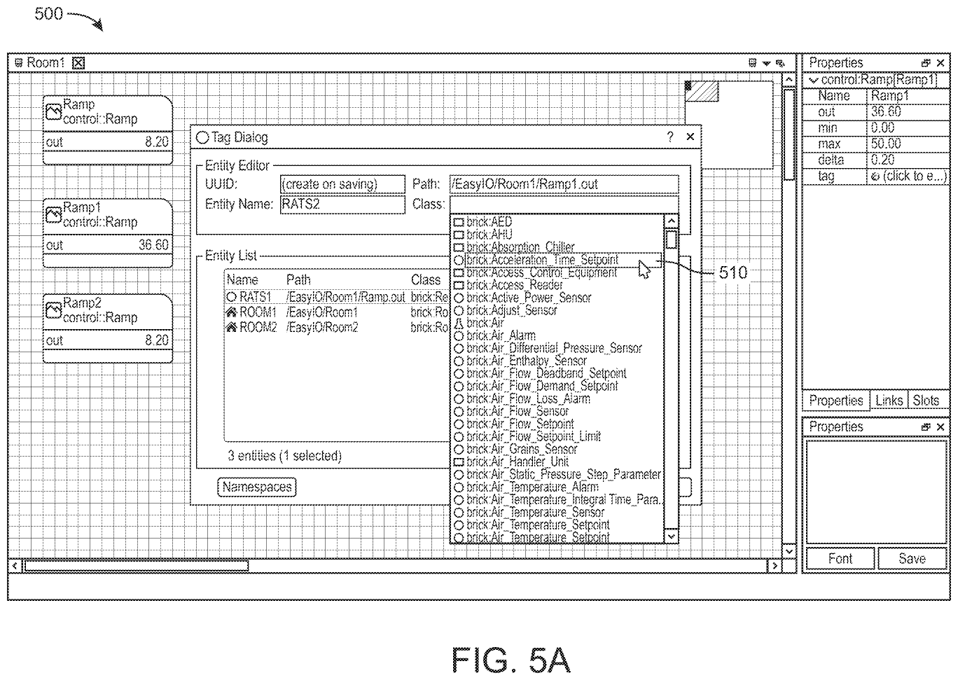

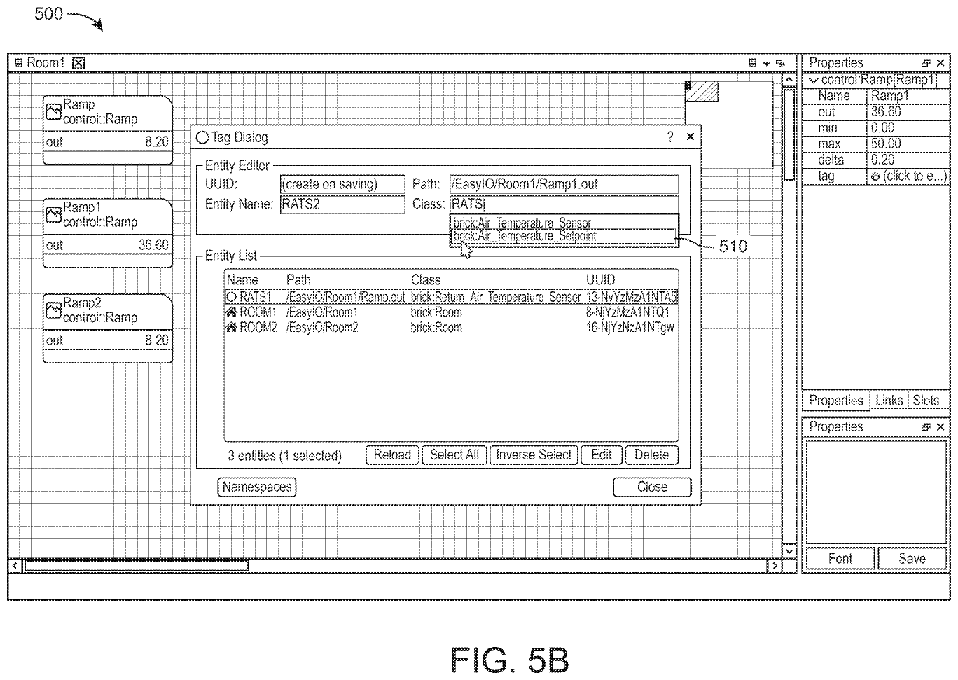

Referring now to FIGS. 5A-5B, GUI 500 for suggesting tags is shown, according to an exemplary embodiment. In various embodiments, GUI 500 is displayed in response to a user selection of tag 422 in GUI 400. In various embodiments, GUI 500 includes a tag dialog to facilitate assigning properties to an entity such as assigning a semantic tag to a digital representation of a piece of HVAC equipment. In various embodiments, GUI 500 includes a section to select tag 510 such as a semantic tag. In various embodiments, GUI 500 displays a list of tags from which a user can select tag 510. In some embodiments, GUI 500 suggests one or more tags (e.g., suggested tags) based on context information associated with the entity. For example, BAS controller 202 may perform semantic extraction on a string name of an entity to identify one or more keywords that may be used to search a list of possible tags and surface a subset of the possible tags to the user corresponding to tags that are likely to be associated with the entity based on the entity name. Additionally or alternatively, GUI 500 may facilitate a user to enter text and GUI 500 may surface tags associated with the text. For example, as shown in FIG. 5B, a user may enter “RATS” and GUI 500 may surface a first tag “Air Temperature Sensor” and a second tag “Air Temperature Setpoint.” In various embodiments, a user may select tag 510 to be assigned to the entity. For example, a user may select tag 510 from a list of tags and BAS controller 202 may assign tag 510 to the entity (e.g., by updating a graph data structure, etc.). In various embodiments, BAS controller 202 automatically generates a GUI element in response to a data point and/or an entity being tagged with metadata. For example, BAS controller 202 may automatically update a user interface associated with a tagged entity to add sensor measurements generated by the tagged entity to the user interface.

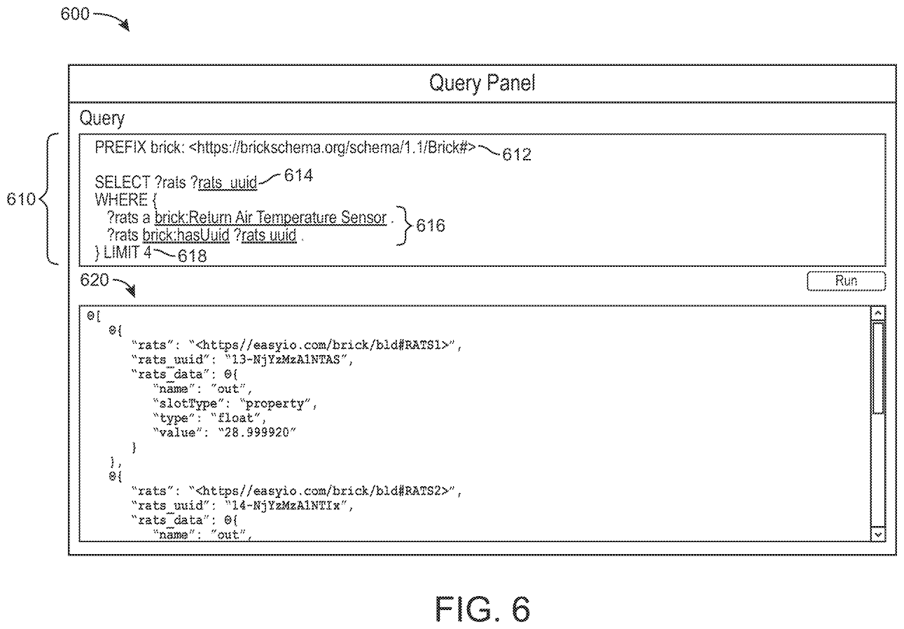

Referring now to FIG. 6, GUI 600 for executing queries based on semantic tags is shown, according to an exemplary embodiment. In various embodiments, GUI 600 facilitates querying a database such as a digital twin of a building to generate user interface elements to dynamically visualize data such as sensor measurements. For example, a user may generate a query via GUI 600 to query an EMR to retrieve and display data related to an entity such as a real time pulse oximeter measurement from a patient. Additionally or alternatively, GUI 600 may facilitate querying a database to retrieve information associated with entities. For example, a user may retrieve various parameters associated with entities sharing a tag such as sensor measurements of sensor entities having a “VAV” tag. In various embodiments, GUI 600 facilitates retrieving information such as data points for use in an operation. For example, a user using GUI 600 may quickly and efficiently retrieve a number of data points associated with a semantic description for use in training a machine learning model. As another example, GUI 600 may be used to retrieve all sensor measurements associated with a room as an input to a fault prediction system. In some embodiments, GUI 600 is displayed via an online interface. For example, a user may connect to a newly installed device controller via an IP address of the device controller and may view GUI 600 to query information associated with devices controller by the device controller. In various embodiments, GUI 600 includes query interface 610 and output 620. In various embodiments, a user may input a query via query interface 610. For example, a user may generate a query using a programming language such as structured query language (SQL) to retrieve data from a data structure such as a graph data structure and generate a user interface element to display the retrieved data. As another example, a user may generate a SPARQL query.

In various embodiments, query interface 610 supports flexible query languages. For example, query interface 610 may support multiple query formats including custom query formats. As shown, a query may include format identifier 612, selection identifier 614, location identifier 616, and size identifier 618. In various embodiments, format identifier 612 facilitates specifying a query format. For example, a user may specify a naming format associated with tags queried using query interface 610 such as a Brick Schema format. In various embodiments, selection identifier 614 facilitates specifying an attribute/property of an entity to be displayed. For example, a user may specify that a user interface element associated with a retrieved entity should be identified using the unique identifier associated with the entity. In various embodiments, selection identifier 614 facilitates identifying which entities and/or tags are selecting via query interface 610. For example, a user may specify that all entities having tags including the substring “VAV” should be selected. In various embodiments, location identifier 616 may facilitate identifying a location for the query to search. For example, a user may specify that the query should search all entities having the tag “Return_Air_Temperature_Sensor.” In various embodiments, location identifier 616 facilitates multiple inputs. For example, a user may input multiple search parameters separated by an “OR” or an “AND” operator to further define the search criteria of a query. In various embodiments, size identifier 618 facilitates specifying a limit on the number of entities to return via the query. For example, a user may specify that only four entities should be returned via the query (e.g., to prevent an overload of results, etc.).

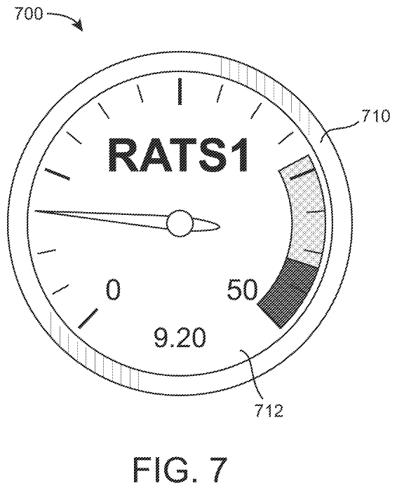

In various embodiments, results of query entered via query interface 610 are displayed via output 620. Output 620 may display structured data representing entities retrieved based on the query parameters of query interface 610. Additionally or alternatively, GUI 600 may display query results via one or more user interface elements described in greater detail below with reference to FIG. 7. In various embodiments, output 620 facilitates modifying query parameters. For example, a user may view a query output via output 620 and may adjust one or more query parameters to identify specific entities. In various embodiments, output 620 includes an entity identifier and any associated entity data. In some embodiments, output 620 includes a link to view the entity. For example, output 620 may include a link to access a GUI with details of the entity.

Referring now to FIG. 7, GUI element 700 for displaying information according to a query is shown, according to an exemplary embodiment. In various embodiments, GUI element 700 is displayed in response to a user query via GUI 600. For example, a user may submit a query via GUI 600 and BAS controller 202 may generate one of GUI element 700 for each entity and/or datapoint returned by the query. In various embodiments, GUI element 700 includes a widget shown as dial 710. Dial 710 may display data associated with an identified entity such as a sensor. It should be understood that other widgets are possible. In various embodiments, each widget dynamically references data from a tagged entity such that the widget dynamically updates based on changes in the entity (e.g. to update sensor measurements, etc.). In various embodiments, dial 710 is a dynamic meter to display HVAC equipment parameters. Additionally or alternatively, dial 710 may include a dynamic meter to display healthcare data such as a patient heartrate. In various embodiments, BAS controller 202 generates dial 710 based on queries such as queries to select one or more entities associated with a particular tag. In various embodiments, dial 710 may be implanted within a user interface to display trend data associated with an entity. In various embodiments, BAS controller 202 customizes dial 710 based on the data being displayed. For example, if dial 710 is displaying Kwh energy data then BAS controller 202 may scale dial 710 according to the data being displayed and add a unit of Kwh. In various embodiments, dial 710 includes data 712. Data 712 may display a value. For example, data 712 may display a numerical value associated with a sensor measurement identified via a query.

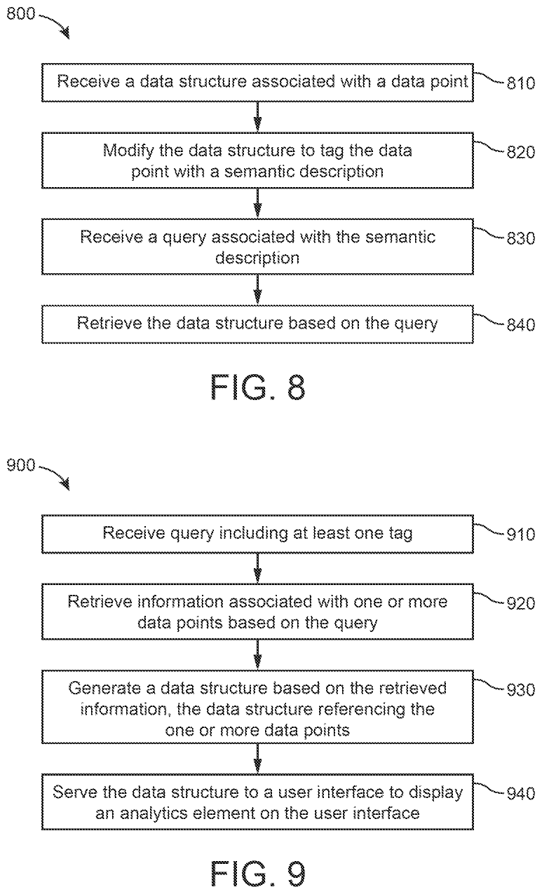

Referring now to FIG. 8, method 800 for tagging a data point and retrieving information based on a tag is shown, according to an exemplary embodiment. In various embodiments, BAS controller 202 performs method 800. For example, BAS controller 202 may tag an entity and/or a data point to facilitate identifying the entity and/or data point with a query. In various embodiments, method 800 facilitates a user friendly manner of adding trend data to a user interface. For example, a user wishing to add HVAC performance metrics to a user interface may generate a query to quickly retrieve the HVAC performance metrics using tags and generate user interface elements to display the HVAC performance metrics on the user interface. At step 810, BAS controller 202 may receive a data structure associated with a data point. In various embodiments, step 810 includes retrieving information from a graph data structure. For example, BAS controller 202 may display a GUI including a digital representation of a piece of HVAC equipment to facilitate a user to view and/or modify tags associated with the digital representation of the piece of HVAC equipment.

At step 820, BAS controller 202 may modify the data structure to tag the data point with a semantic description. In various embodiments, step 820 includes receiving a semantic description from a user to apply to the data point. It should be understood that while method 800 is described in relation to data points, method 800 may also apply to tagging entities. In various embodiments, step 820 includes suggesting one or more tags for a data point to a user. For example, BAS controller 202 may receive a partial string from a user and may use the partial string to surface tags associated with an entity according to the partial string such as surfacing “Return_Air_Temperature_Sensor” based on an input of “RATS.” In some embodiments, BAS controller 202 may automatically generate a user interface element to display data associated with a data point in response to the data point being tagged. For example, a user interface dashboard displaying information associated with HVAC equipment may be automatically updated to include a user interface element displaying sensor measurements in response to a digital representation of the sensor being tagged with metadata.

At step 830, BAS controller 202 may receive a query associated with the semantic description. For example, BAS controller 202 may receive a query inputted by a user via a GUI for all data points associated with the tag “Return_Air_Temperature_Sensor.” In various embodiments, the query is a partial query. For example, BAS controller 202 may receive a query of “SATS” associated with a tag of “Supply_Air_Temperature_Sensor.” At step 840, BAS controller 202 may retrieve the data structure based on the query. In various embodiments, step 840 includes retrieving information associated with data point based on the query. For example, BAS controller 202 may retrieve a sensor measurement associated with a data point tagged with the semantic description. In various embodiments, step 840 includes displaying retrieved data to a user. For example, BAS controller 202 may display information associated with a retrieved entity and/or data point to a user via a GUI.

Referring now to FIG. 9, method 900 for serving an analytics element associated with a tag is shown, according to an exemplary embodiment. In various embodiments, BAS controller 202 performs method 900. Method 900 may facilitate adding trend data to user interfaces. For example, a user may quickly identify trend data such as power consumption information from a database using tags associated with the power consumption information (e.g., tags associated with sensors that measure power consumption, etc.), may generate a user interface element such as a dynamic display dial, and may automatically embed the dynamic display dial in a user interface via method 900. At step 910, BAS controller 202 may receive a query including at least one tag. In various embodiments, the tag is formatting according to a known formatting such as the Brick Schema. Additionally or alternatively, the query may be formatted in a different manner such as in shorthand. For example, a user may submit a query including the shorthand “SATS” to identify data points and/or entities with the tag “Supply_Air_Temperature_Sensor.” In some embodiments, the query includes multiple tags. The tags may be associated with entities such as spaces, equipment, people, or events. Additionally or alternatively, the tags may be associated with data points such as sensor measurements. In some embodiments, the tags are associated with EMR data such as tags associated with medical diagnoses.

At step 920, BAS controller 202 may retrieve information associated with one or more data points based on the query. For example, BAS controller 202 may execute the query on a data structure stored in a database to identify information according to the query parameters. For example, BAS controller 202 may receive a query to identify air temperature measurements associated with a particular zone and may search a database storing data in a resource description framework (RDF) formatting to identify sensors based on the query. In various embodiments, step 920 includes retrieving a digital representation of an entity. For example, BAS controller 202 may retrieve a digital representation of a sensor having associated sensor measurements. In some embodiments, step 920 includes retrieving a data point such as a set of timeseries data associated with a sensor.

At step 930, BAS controller 202 may generate a data structure based on the retrieved information, the data structure referencing the one or more data points. In various embodiments, the data structure includes a dynamic display such as a user interface element that is linked to the data associated with the one or more data points. In various embodiments, the data structure includes a GUI element. In various embodiments, BAS controller 202 dynamically customizes the GUI element based on the information being displayed. For example, the GUI element may include a dial, a histogram, a map, a bar graph, a pie chart, a heatmap, a scatterplot, a line-graph, a box-and-whiskers plot, and/or the like.

At step 940, BAS controller 202 may serve the data structure to a user interface to display an analytics element on the user interface. For example, BAS controller 202 may embed a GUI element in a user interface to display trend data associated with an entity. In some embodiments, step 940 includes generating a GUI element to display sensor measurements. For example, a user may generate a GUI element to display sensor measurements associated with a piece of HVAC equipment and embed the GUI element in a dashboard. In various embodiments, BAS controller 202 automatically generates and embeds the GUI element based on a user query. Additionally or alternatively, step 940 may include performing other operations. For example, step 940 may include transmitting the information retrieved as part of step 920 to an external system to facilitate a fault determination. As another example, step 940 may include updating a model of a space based on the retrieved information. In some embodiments, step 940 includes generating a control message to operate a device. For example, BAS controller 202 may receive trend data associated with an air temperature sensor and may generate a control message to operate a VAV box. In some embodiments, step 940 includes controlling an energy usage associated with a space. Additionally or alternatively, step 940 may include training a machine learning model associated with a space based on the retrieved information. For example, BAS controller 202 may retrieve a number of tagged data points and may train a machine learning model for fault prediction using tags associated with the tagged data points as a classifier for the training data. It should be understood that other operations using the retrieved information not explicitly mentioned herein are possible and within the scope of the present disclosure.

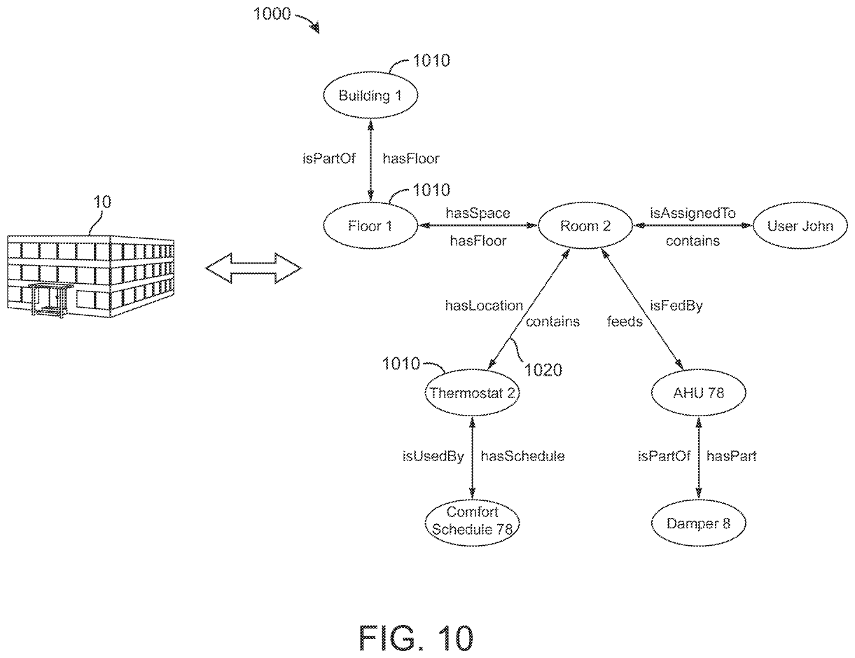

Referring now to FIG. 10, entity graph 1000 is shown, according to an exemplary embodiment. In various embodiments, BAS controller 202 represents building 10 as entity graph 1000. For example, BAS controller 202 may generate a digital representation for a building and may use the digital representation as a data analytics model to perform various functions. In various embodiments, entity graph 1000 includes one or more data points having tags. For example, a data point may be represented as a node and a tag may be represented as an edge connecting two nodes. In brief overview, entity graphs such as entity graph 1000 are structured data stored in memory (e.g., a database, etc.). Entity graph 1000 may include digital twins. Digital twins may be digital representations of real world spaces, equipment, people, and/or events. In various embodiments, digital twins represent buildings, building equipment, people associated with buildings, and/or events associated with buildings (e.g., buildings 10, etc.). An entity graph may include nodes and edges, where each node of the entity graph represents an entity and each edge is directed (e.g., from a first node to a second node) and represents a relationship between entities (e.g., indicates that the entity represented by the first node has a particular relationship with the entity represented by the second node). For example, an entity graph may be used to represent a digital twin of a person.

Entities can be things and/or concepts related to spaces, people, and/or asset. For example, the entities could be “B7F4 North”, “Air Handling Unit,” and/or “meeting room.” The nodes can represent nouns while the edges can represent verbs. For example, the edges can be “isA,” “hasPart,” and/or “feeds.” In various embodiments, the edges represent relationships. While the nodes represent the building and its components, the edges describe how the building operates. The nodes and edges together create a digital twin of a particular building. In some embodiments, the entities include properties or attributes describing the entities (e.g., a thermostat may have a particular model number attribute). The components of the entity graph form large networks that encode semantic information for a building.

The entity graph is configured to enable flexible data modeling for advanced analytics, control, and/or artificial intelligence applications, in some embodiments. These applications may require, or benefit from information modeling including interconnected entities. Other data modeling techniques based on a table, a hierarchy, a document, and/or a relational database may not be applicable. The entity graph can be a foundational knowledge management layer to support other higher level applications, which can be, complex root cause, impact analysis, building powerful recommendation engines, product taxonomy information services, etc. Such a multilayer system, a system of system topologies, can benefit from an underlying entity graph.

The entity graph can be a data contextualization layer for all traditional and/or artificial intelligence applications. The entity graph can be configured to capture evidence that can be used to attribute the strengths of entity relationships within the entity graph, providing the applications which utilize the entity graph with context of the systems they are operating. Without context (e.g., who the user is, what the user is looking for, what the target of a user request is, e.g., find a meeting room, increase a temperature in my office) these applications may never reach their full potential. Furthermore, the entity graph provides a native data structure for constructing question and answer type systems, e.g., a chatbot, that can leverage and understand intent.

In various embodiments, the entity graph includes data from various sources. For example, the entity graph may include data associated with people, places, assets, and/or the like. In various embodiments, the data source(s) represent a heterogenous source data schema such as an open source common data model (e.g., a Brick Schema/extensions, etc.).

In various embodiments, entity graph includes digital twins and/or context information. A digital twin is a digital representation of spaces, assets, people, events, and/or anything associated with a building or operation thereof. In various embodiments, digital twins are modeled in the entity graph. In various embodiments, digital twins include an active compute process. For example, a digital twin may communicate with other digital twins to sense, predict and act. In various embodiments, digital twins are generated dynamically. For example, a digital twin corresponding to a conference room may update its status by looking at occupancy sensors or an electronic calendar (e.g., to turn its status “available” if there is no show, etc.). In various embodiments, digital twins and/or the entity graph include context information. Context information may include real-time data and a historical record of each system in the environment (e.g., campus, building, facility, space, etc.). Context information may be stored in the entity graph. In various embodiments, context information facilitates flexible data modeling for advanced analytics and AI application in scenarios that model highly interconnected entities.

The entity graph may not be a configuration database but may be a dynamic representation of a space, person, event, and the like. The entity graph can include operational data from entities which it represents, e.g., sensors, actuators, card access systems, occupancy of a particular space, thermodynamics of the space as a result of actuation, etc. The entity graph can be configured to continually, and/or periodically, ingest new data of the space and thus the entity graph can represent a near real-time status of cyber-physical entities and their inter-relationships. For this reason, artificial intelligence can be configured to introduce a virtual entity and new semantic relationships among entities, in some embodiments.

The entity graph is configured to facilitate adaptive controls, in some embodiments. The entity graph can be configured to adapt and learn over time. The entity graph can be configured to enable dynamic relationships between building information and other facility and enterprise systems to create new insights and drive new optimization capabilities for artificial intelligence systems. As relationships can be learned over time for the entity graph, the artificial intelligence systems and also learn overtime based on the entity graph.

Entity graph 1000 includes entities 1010 (stored as nodes within entity graph 1000) describing spaces, equipment, events, and people (e.g., business employees, etc.). In various embodiments, entities 1010 are associated with or otherwise include agents (e.g., agents may be assigned to/associated with entities, etc.). Additionally or alternatively, agents may be represented as nodes in entity graph 1000 (e.g., agent entities, etc.). Furthermore, edges 1020 are shown between entities 1010 directionally describing relationships between two of entities 1010 (stored as edges within entity graph 1000). In various embodiments, BAS controller 202 may traverse entity graph 1000 to retrieve a description of what types of actions to take for a certain device, what the current status of a room is (e.g., occupied or unoccupied), etc.

As an example, entity graph 1000 illustrates a building called “Building 1.” Building 1 has a directional relationship to a floor called “Floor 1.” The relationship may be an edge “hasFloor” indicating that the building (e.g., the building represented by entity 1010) has a floor (e.g., the floor represented by entity 1010). Furthermore, a second edge “isPartOf” from Floor 1 to Building 1 indicates that the floor (e.g., the floor represented by entity 1010) is part of Building 1 (e.g., the building represented by entity 1010).

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

The present disclosure contemplates methods, systems and program products on any machine-readable media for accomplishing various operations. The embodiments of the present disclosure can be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures show a specific order of method steps, the order of the steps may differ from what is depicted. Also two or more steps can be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

What is claimed is:

1. A method of retrieving data using metadata tags, comprising:
identifying, by a processing circuit from a data structure, a digital representation of a device deployed within a space;
tagging, by the processing circuit, a data point associated with the digital representation of the device with a semantic description having a tag schema;
receiving, by the processing circuit, a query including a partial string referencing the tag schema;
identifying, by the processing circuit, the semantic description from a plurality of semantic descriptions based on the partial string of the query and the tag schema;
retrieving, by the processing circuit, one or more tagged data points by querying the data structure using the semantic description; and
automatically performing an operation using the one or more tagged data points.

2. The method of claim 1, wherein automatically performing the operation includes:
generating, by the processing circuit, a user interface element to display real time trend data associated with the retrieved one or more tagged data points; and
automatically embedding, by the processing circuit, the user interface element in a user interface.

3. The method of claim 2, wherein the real time trend data includes at least one of an alarm status or a sensor measurement value.

4. The method of claim 2, wherein the data structure includes an electronic medical record (EMR) and wherein the real time trend data includes at least one of a patient health status or a biological measurement associated with a patient.

5. The method of claim 2, wherein retrieving the one or more tagged data points, generating the user interface element, and automatically embedding the user interface element are performed automatically in response to identifying the semantic description.

6. The method of claim 2, further comprising automatically formatting, by the processing circuit, at least one of a units or a display scale of the user interface element based on the real time trend data.

7. The method of claim 1, wherein the data point is stored using a resource description framework (RDF) format.

8. The method of claim 1, wherein the data structure includes a digital twin representing at least one of a space, a person, a piece of equipment, or an event.

9. The method of claim 1, wherein automatically performing the operation includes at least one of: (i) determining a fault associated with the space based on the one or more tagged data points, (ii) generating a predictive control model for the space based on the one or more tagged data points, (iii) generating a control message to control the device based on the one or more tagged data points, (iv) controlling an energy usage associated with the space based on the one or more tagged data points, (v) training a machine learning model associated with the space using the one or more tagged data points, (vi) updating a model associated with the space based on user feedback corresponding to the one or more tagged data points, or (vii) updating an architectural model for the space based on the one or more tagged data points.

10. A controller for managing building equipment, comprising:
a processing circuit including a processor and memory, the memory storing instructions thereon that, when executed by the processor, causes the processing circuit to:
tag a data point stored in a data structure with a semantic description having a tag schema, the data point associated with a digital representation of the controller;
receive a query including a partial string referencing the tag schema;
identify the semantic description from a plurality of semantic descriptions based on the partial string of the query and the tag schema;
retrieve one or more tagged data points by querying the data structure using the semantic description; and
automatically perform an operation using the one or more tagged data points.

11. The controller of claim 10, wherein automatically performing the operation includes:
generating a user interface element to display real time trend data associated with the retrieved one or more tagged data points; and
automatically embedding the user interface element in a user interface.

12. The controller of claim 11, wherein the real time trend data includes at least one of an alarm status or a sensor measurement value.

13. The controller of claim 11, wherein retrieving the one or more tagged data points, generating the user interface element, and automatically embedding the user interface element are performed automatically in response to identifying the semantic description.

14. The controller of claim 11, wherein the instructions further cause the processing circuit to automatically format at least one of a units or a display scale of the user interface element based on the real time trend data.

15. The controller of claim 10, wherein the data point is stored using a resource description framework (RDF) format.

16. The controller of claim 10, wherein the data structure includes a digital twin representing at least one of a space, a person, a piece of equipment, or an event.

17. The controller of claim 10, wherein automatically performing the operation includes collaboratively in association with an external system at least one of: (i) determining a fault associated with the space based on the one or more tagged data points, (ii) generating a predictive control model for the space based on the one or more tagged data points, (iii) generating a control message to control the device based on the one or more tagged data points, (iv) controlling an energy usage associated with the space based on the one or more tagged data points, (v) training a machine learning model associated with the space using the one or more tagged data points, (vi) updating a model associated with the space based on user feedback corresponding to the one or more tagged data points, or (vii) updating an architectural model for the space based on the one or more tagged data points.

18. One or more non-transitory computer-readable storage media having instructions stored thereon that, when executed by one or more processors, cause the one or more processors to:
identify, within a data structure, a digital representation of a device deployed within a space;
tag a data point associated with the digital representation of the device with a semantic description having a tag schema;
receive a query including a partial string referencing the tag schema;
identify the semantic description from a plurality of sematic descriptions based on the partial string of the query and the tag schema;
retrieve one or more tagged data points by querying the data structure using the semantic description; and
automatically performing an operation using the one or more tagged data points.

19. The one or more non-transitory computer-readable storage media of claim 18, wherein automatically performing the operation includes:
generating a user interface element to display real time trend data associated with the retrieved one or more tagged data points; and
automatically embedding the user interface element in a user interface.

20. The one or more non-transitory computer-readable storage media of claim 18, wherein automatically performing the operation includes at least one of: (i) determining a fault associated with the space based on the one or more tagged data points, (ii) generating a predictive control model for the space based on the one or more tagged data points, (iii) generating a control message to control the device based on the one or more tagged data points, (iv) controlling an energy usage associated with the space based on the one or more tagged data points, (v) training a machine learning model associated with the space using the one or more tagged data points, (vi) updating a model associated with the space based on user feedback corresponding to the one or more tagged data points, or (vii) updating an architectural model for the space based on the one or more tagged data points.
